(19) United States
(12) Patent Application Publication
(10) Pub. No.: US 2020/0093626 A1
(43) Pub. Date: Mar. 26, 2020

(54) ORTHOTIC DEVICES AND METHODS OF USING THE SAME

(71) Applicant: Georgia Tech Research Corporation, Atlanta, GA (US)

(72) Inventors: Geza F. Kogler, Atlanta, GA (US); Omer Inan, Atlanta, GA (US); Nick Bolus, Atlanta, GA (US); Caitlin Teague, Atlanta, GA (US)

(21) Appl. No.: 16/494,446

(22) PCT Filed: Mar. 23, 2018
(86) PCT No.: PCT/US2018/023978

§ 371 Date: Sep. 16, 2019

Related U.S. Application Data
(60) Provisional application No. 62/475,442, filed on Mar. 23, 2017.

(51) Int. Cl. A61F 5/01

(52) U.S. Cl. CPC A61F 5/0113; A61F 2005/0179; A61F 2005/0165

(57) ABSTRACT

An orthotic device is disclosed. The orthotic device can comprise a passive orthosis and at least one sensor. At least a first portion of the orthosis can be configured to be positioned on a first side of a joint of a user. At least a second portion of the orthosis can be configured to be positioned on a second side of the joint of a user. The at least one sensor can be coupled to the orthosis and configured to collect data indicative of a user's response to a resistive force applied to the joint.

### CROSS-REFERENCE TO RELATED APPLICATIONS

This application claims the benefit of U.S. Provisional Application Ser. No. 62/475,442, filed 23 Mar. 2017, which is incorporated herein by reference in its entirety as if fully set forth below.

### STATEMENT REGARDING FEDERALLY SPONSORED RESEARCH

This invention was made with U.S. Government support under Grant No. W81XWH-15-1-0479 awarded by the by the U.S. Army. The U.S. Government has certain rights in the invention.

### TECHNICAL FIELD

The present invention relates generally to an orthotic device and corresponding method for using same. More specifically, the present invention relates to a wearable passive orthosis capable of measuring a user's response to a resistive force applied to a joint.

### BACKGROUND

Orthoses—wearable devices used to control joint motion and provide corrective support for, or improve the functionality of, impaired limbs/joints—provide a simple, non-operative, inexpensive, and effective treatment for myriad neuromuscular and musculoskeletal disorders. The most commonly prescribed orthosis is an ankle-foot orthosis (AFO). In individuals with a loss of volitional lower limb motor function, an AFO can provide the necessary stability for walking and standing to maximize functional mobility for the user. The inability to dorsiflex the foot (i.e., foot drop) is one of the most common lower limb conditions and is associated with peripheral nerve injury, stroke, diabetes, and an array of neurological disorders such as multiple sclerosis and Charcot-Marie-Tooth disease. With reduced ability to lift the foot up towards the shin and thus achieve toe clearance during the swing phase of gait, users with foot drop often compensate for this deficit, leading to attendant degeneration of normal gait mechanics, which can result in both higher metabolic cost of walking and heightened risk of tripping and falling. Prescription of lower-limb orthoses is recommended especially for the elderly due to increased need for stability and protection against failing-induced injury. With the rising aging population and incidence of stroke, the burden on the healthcare system—and in particular, the orthotics and orthopedics communities—will undoubtedly grow heavier, presenting a need for more effective, affordable, and personalized home-use orthoses to improve user mobility, safety, and device adoption.

AFOs have generally been shown to improve locomotor function (e.g., gait velocity, stride length, walking efficiency, balance) and mitigate injury risk in hemiplegic gait. However, several studies have demonstrated that, if the devices are not designed or fit properly, such improvements can be relatively insignificant and can in fact cause discomfort and further compromise gait mechanics, perhaps leading to muscle disuse (and eventual atrophy) and reduced user acceptance of the intervention. Presently the methods associated with determining the proper magnitude of orthotic constraint for an AFO are qualitative and subjective. An orthotist typically relies on experiential clinical estimates of the AFO's stiffness by manually deflecting the orthosis, thereby assessing whether the device offers the requisite corrective forces to overcome the motor deficit (e.g., foot drop). The desired treatment outcome of “toe clearance during swing phase” is then clinically confirmed by observation gait analysis. Since the improvements in gait are generally so apparent when adequate resistance to foot drop—and thus swing-phase toe clearance—are achieved, little attention has been devoted to the optimization of orthotic resistance/assistance nor to the functional consequences of varying the degree of device stiffness, despite studies which have suggested that user-matching of orthotic constraint can significantly influence individual outcomes. Further, the advances in the art include active AFOs (i.e., AFO's in which an external power source is employed to actuate the ankle joint movement) and adaptive orthoses to assist individuals with movement disorders, employing techniques such as biofeedback via functional electrical stimulation, actuated robotic assistance, and variable-impedance joints. While these devices represent an improvement in the art, they are bulky, complicated, expensive, and unconducive to mass production, making them presently unsuitable for home use.

Consequently, in the art, there lacks an objective way to observe and quantify various parameters of gait in the clinic in order to identify an optimal set of orthosis properties for each user, which may depend on the degree of paresis, paralysis, spasticity of the lower limb as well as his/her anthropometrics and capacity for recovery. To address these shortcomings, various embodiments of the present invention relate to passive orthosis (i.e., an orthotic device in which no external power source is employed to actuate joint movement), which can be fully wearable and portable, and can allow a clinician/researcher to modulate joint stiffness of the orthosis, study a user's biomechanical response to such perturbation, and ultimately identify and prescribe an optimal orthosis stiffness on a user-specific basis.

### SUMMARY

An orthotic device and corresponding method for using same is described.

In some embodiments, an orthotic device comprises a passive orthosis and at least one sensor. The passive orthosis may include a first portion configured to be positioned on a first side of a joint of a user. The passive orthosis may also include a second portion configured to be positioned on a second side of the joint of a user. The at least one sensor may be coupled to the orthosis and configured to collect data indicative of a user's response to a resistive force applied to the joint. In some embodiments, the at least one sensor may be integral to or embedded in the orthosis. In some embodiments, the at least one sensor may be coupled to an external portion of the orthosis

In some embodiments, the data indicative of a user's response to a resistive force applied the joint, collected by the at least one sensor, may include data indicative of foot-ground placement of the user during a movement by the user. The foot-ground placement may be indicative of whether the user performs a heel-toe strike or toe-heel strike.

In some embodiments, the data indicative of a user's response to a resistive force applied the joint, collected by the at least one sensor, may include data indicative of a range of motion of the joint during a movement by the user.

In some embodiments, the data indicative of a user's response to a resistive force applied the joint, collected by the at least one sensor, may include data indicative of pressures applied to the orthosis by portions of the user during a movement by the user. The pressures applied to the orthosis by portions of the user during a movement by the user can include, but are not limited to, pressures on the anterior/proximal calf, posterior proximal calf, dorsum of the foot, and/or plantar foot.

In some embodiments, the at least one sensor may be configured to collect data indicative of a user's response to a resistive force applied to the joint at multiple times during a movement by the user.

In some embodiments, the orthotic device may comprise at least one motion resisting mechanism configured to restrict movement of the joint in at least one direction. The at least one motion resisting mechanism comprises at least one spring. The at least one second sensor may be configured to collect data indicative of a force applied by the resisting mechanism. In some embodiments, the at least one second sensor may be a force/torque sensor.

In some embodiments, the at least one motion resisting mechanism may be adjustable (e.g., length and/or girth) to vary a magnitude of a force applied by the at least one motion resisting mechanism.

In some embodiments, the at least one motion resisting mechanism may be coupled to the orthosis on an anterior side of the first side of a joint of a user. The orthotic device may include a reaction arm coupled to the orthosis, the reaction arm in mechanical communication with the at least one motion resisting mechanism.

In some embodiments, the reaction arm may be adjustable between a first position in which the at least one motion resisting mechanism is configured to restrict movement of the joint in at least a first direction and a second position in which the motion resisting mechanism is configured to restrict movement of the joint in at least a second direction.

In some embodiments, the orthosis is modular and adjustable to fit a plurality of users.

In some embodiments, the orthotic device may include a transmitter coupled to the orthosis, the transmitter configured to output at least a portion of the data collected by the at least one sensor to a second device remote from the orthotic device.

In some embodiments, the orthosis may the orthosis include at least one stirrup having a first end and a second end, the first end coupled to the orthosis at a position proximate the joint of the user, the second end coupled to the orthosis at a position proximate the first side of the joint of the user.

In some embodiments, the orthosis may include at least one closure/suspension configured to secure the first side of the joint of the user to the orthosis. The at least one sensor may be coupled to the at least one closure/suspension.

In some embodiments, the orthotic device may be wearable by the user.

In some embodiments, the at least one sensor of the orthotic device may comprise a reaction force or torque sensor.

In some embodiments, the at least one sensor of the orthotic device may comprise a force sensitive resistor.

In some embodiments, the at least one sensor of the orthotic device may comprise a contact sensor.

In some embodiments, the at least one sensor of the orthotic device may comprise an optical encoder.

In some embodiments, the at least one sensor of the orthotic device may comprise a sensor that quantifies angular motion.

In some embodiments, a method for collecting data indicative of a user's response to a resistive force applied to a joint of the user, the user wearing a passive orthosis positioned about the joint, the passive orthosis comprising at least one sensor includes collecting data with the at least one sensor of the passive orthosis, the data indicative of the user's response to a resistive force applied to the joint.

In some embodiments, the method for collecting data indicative of a user's response to a resistive force applied to a joint of the user includes storing the collected data on a memory device of the orthosis.

In some embodiments, the method for collecting data indicative of a user's response to a resistive force applied to a joint of the user includes transmitting at least a portion of the collected data to a remote device. The transmittal of at least a portion of the collected data to remote device may be performed in real-time as the user moves the joint.

In some embodiments, the method for collecting data indicative of a user's response to a resistive force applied to a joint of the user may also include generating, on the remote device, a graphical representation of the at least a portion of the data collected by the at least one sensor.

In some embodiments, the orthotic device includes a passive orthosis, at least one sensor coupled to the orthosis, and at least one motion resisting mechanism. A first portion of the orthosis may be configured to be secured to a lower leg of a user between an ankle and a knee of the user, and wherein a second portion of the orthosis is configured to be secured to a foot of the user. The at least one sensor coupled to the orthosis may be configured to collect data indicative of the user's response to a resistive force applied to the ankle. The at least one motion resisting mechanism may be configured to restrict movement of the ankle in at least one of a plantarflexion direction and a dorsiflexion direction by applying the resistive force to the ankle.

Other aspects and advantages of the invention can become apparent from the following drawings and description, all of which illustrate the principles of the invention, by way of example only.

### BRIEF DESCRIPTION OF THE DRAWINGS

The various aspects of the invention described above may be better understood by referring to the following description taken in conjunction with the accompanying drawings. The drawings are not necessarily to scale, emphasis instead generally being placed upon illustrating the principles of the invention.

FIG. 1A is a front perspective view of an instrumented orthotic device positioned on the lower leg of a user according to an embodiment of the invention.

FIG. 1B is a perspective view of an instrumented orthotic device positioned on the lower leg of a user according to an embodiment of the invention.

FIG. 1C is a perspective view of an orthotic device according to an embodiment of the present invention.

FIG. 2 is a graph illustrating benchtop testing results: orthotic torque versus angle relationship in both resistance modes for two stiffness conditions according to an embodiment of the invention.

FIG. 3 is a graph illustrating orthotic torque versus ankle angle relationship in plantarflexion resistance gait study in five stiffness conditions according to an embodiment of the invention.

FIG. 4 is a graph illustrating plantarflexion resistance at a plurality of steps according to an embodiment of the invention.

FIG. 5 is a graph illustrating average plantar center-of-pressure location as orthotic resistance increases according to an embodiment of the invention.

FIG. 6 illustrates the geometric parameters used to construct the mathematical model of the torque versus angle relation according to an embodiment of the invention.

FIG. 7 is a graph illustrating model torque versus angle output for a range of calf band height settings according to an embodiment of the invention.

FIG. 8 illustrates an example flow chart of a method for transmitting data collected by at least one sensor of a passive orthosis, the passive orthosis positioned about a joint of a user according to an embodiment of the invention.

### DETAILED DESCRIPTION

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

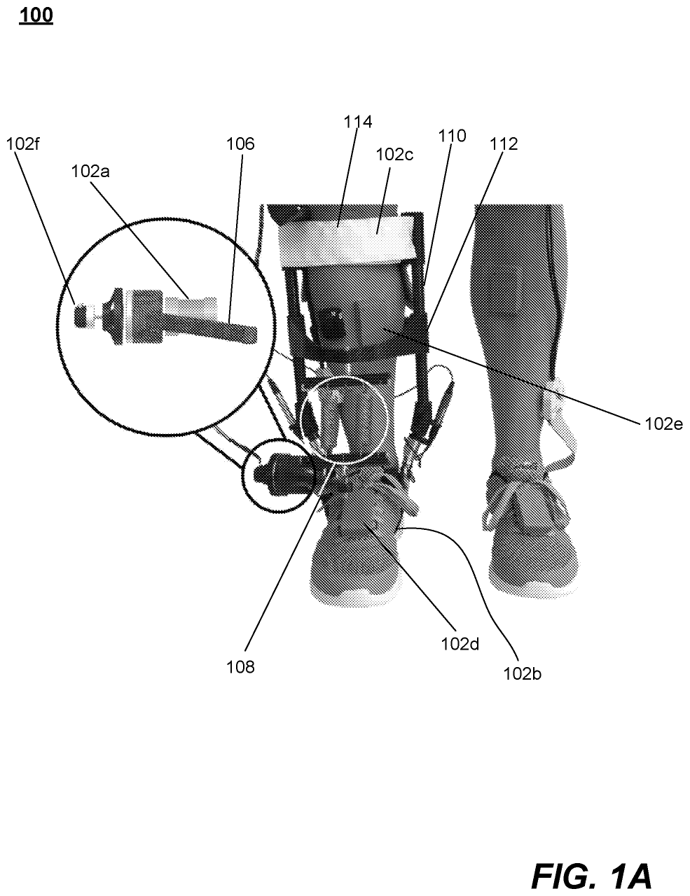

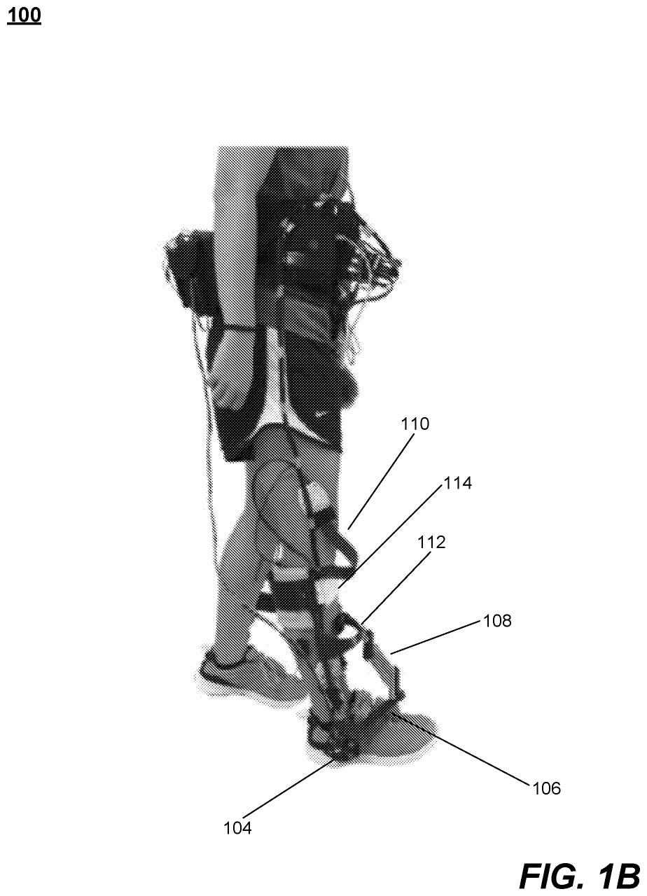

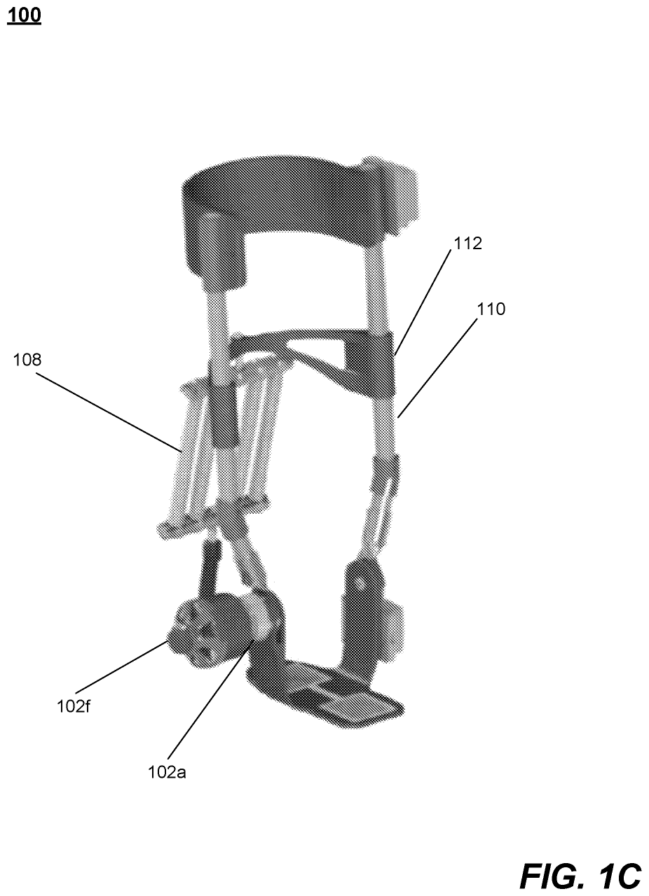

In some embodiments, the present invention provides an orthotic device 110 comprising a passive orthosis 110. As used herein, an orthosis is “passive” if the device does not contain an external power source to actuate movement of a joint by a user. Additionally, the “orthoses” disclosed herein, which aim to enhance a user's existing limb/joint, should not be confused with “prostheses,” which aim to replace a user's existing limb/joint. As shown in FIG. 1, the passive orthosis 110 may include a first portion configured to be positioned on a first side of a joint of a user, e.g., lower leg or calf of a user. The passive orthosis 110 may also include a second portion configured to be positioned on a second side of the joint of a user, e.g., foot of a user. Although FIG. 1 depicts the orthotic device 110 in the context of an ankle-foot orthotic device, the invention is not so limited. Rather, the orthotic devices disclosed herein can be applied to many joints of a user, including, but not limited to, ankle, knee, wrist, elbow, shoulder, hip, and the like.

The orthotic device 100 can also include at least one sensor 102a-f. The at least one sensor 102a-f can be many different sensors known in the art, including, but not limited to force sensors, torque sensors, optical encoders, accelerometers, force sensors, initial measurement sensors (IMU), plantar/interface pressure sensors, contact sensors, electromyography sensors (EMG), and the like. In some embodiments, the orthotic device 100 can contain many sensors. The sensor(s) 102a-f can be coupled to the orthosis 110, for example as with sensors 102a-c, 102f, and/or can be positioned proximate the orthosis 110, e.g., attached to the user or otherwise positioned to measure relevant parameters as with sensors 102d-e. In some embodiments, one or more of the sensors 102a-f can be integral or embedded in the orthosis. In some embodiments, one of more of the sensors 102a-f can be coupled to an external portion of the orthosis. The sensor(s) 102a-f can be configured to collect data indicative of a user's response to a resistive force applied to the joint.

In some embodiments, the sensor(s) 102a-f can collect data indicative of a user's response to a variety of resistive forces applied to the joint. As explained below, in some embodiments, the resistive force is applied to the joint by a motion resisting mechanism 108. Thus, the sensor(s) 102a-f can collect data of how a user responds to that force. For example, in some embodiments, the sensor(s) 102a-f can collect data indicative of a resistive force applied to the joint, wherein the first portion of the orthosis is configured to be positioned on a first side of a joint of a user and at least a second portion of the orthosis is configured to be positioned on a second side of the joint of a user. As shown in FIG. 1, the orthosis can include a pressure sensor 102c measuring pressure applied by the user to a portion of the orthosis 110 proximate the user's foot and a pressure sensor 102b measuring pressure applied by the user to a portion or the orthosis 110 proximate the user's shin or calf.

The sensor(s) 102a-f allows the orthotic device 110 to capture a wide range of clinically relevant biomechanical measures. The sensor(s) 102a-f can collect data indicative of a user's response to resistive force and/or range of motion at a plurality of movement positions of the joint of the user. For example, in the case of an ankle-foot orthosis as shown in FIGS. 1A-C, the data can be indicative of one of more of (1) foot-ground placement of the user during a movement (e.g., walking) by the user, e.g., whether the user performs a heel-toe strike or toe-heel strike, (2) a range of motion of the ankle during a movement by the user, and (3) pressures applied to the orthosis by portions of the user during a movement by the user, which may include, but are not limited to, pressures on the anterior/proximal calf, posterior proximal calf, dorsum of the foot, and/or plantar foot. Additionally, in some embodiments, the sensor(s) 102a-f can be configured to collect data at multiple times during a movement by the user, thus, for example, obtaining data indicating the user's response to the resistive force as the ankle is moved through the gait cycle.

The sensor(s) 102a-f and the corresponding parameters they collect may include, but are not limited to: optical encoder measuring ankle rotation; IMU measuring spatial limb orientation; reaction force/torque and strain gage-based measuring orthosis torque; FSR measuring gait states; pressure-sensitive capacitive film measuring plantar and interface pressures; and EMG measuring muscle activity.

The sensor(s) 102a-f may be synchronized to facilitate concurrent analysis. To accomplish synchronization, certain signals (encoder, force/torque sensor, and force sensors) can be recorded by a real-time field programmable gate array-based data acquisition device (e.g., myRIO) at a sampling rate of 1 kHz, while plantar and interface pressures were sampled at 50 Hz, IMU data at 75 Hz, and EMG data at 1926 Hz. The myRIO may be configured to serve as a master triggering device, initiating and terminating acquisition of signals on each sensor system simultaneously at the beginning and end of each data collection trial. The data collected by the sensor(s) 102a-f may be monitored by a remote device in real-time as the user moves the joint. A graphical representation of the at least a portion of the data collected by the at least one sensor may be generated on the remote device.

In some embodiments, the orthotic device 110 further includes a motion resisting mechanism 108. The motion resisting mechanism 108 can be configured to restrict movement of a joint in at least one direction that the joint of the user moves. For example, in the case of an ankle-foot orthosis, the motion resisting mechanism can be configured to restrict movement of the ankle in the plantarflexion or dorsiflexion direction. The motion resisting mechanism 108 can be any motion resisting mechanism known in the art, including but not limited to, springs (e.g., extension, compression, torsion, jack-spring, leaf-spring variety, and the like), mechanical dampers (e.g., linear or rotary variety, including those driven by passive fluid dynamics, hydraulic systems, pneumatics, flexible, and magnetism), or elastic materials (e.g., carbon fiber, aluminum, rubber bands, thermoplastic materials, etc.), and the like. In some embodiments, the motion resisting mechanism is embedded within or part of the orthosis. The configuration of the motion resisting mechanism 108 and the use of motion resisting mechanisms of varying tension may provide modular torsional stiffness or resistance to rotation via interchangeable extension springs coupled either to the anterior and/or posterior to a joint of the user, enabling, for example, the study of the effect of resistance to joint rotation in plantarflexion and dorsiflexion modes independently. For example, a motion resisting mechanism 108 may comprise one or more springs mounted in parallel with each other to provide resistance to ankle rotation in a plantarflexion and/or dorsiflexion direction. Also, the motion resisting mechanism may be configured to provide different amounts of force to the joint of a user. In particular, this feature can provide an accurate measurement of the amount of stiffness required of an orthosis to be fitted to the user.

The motion resisting mechanism 108 can be positioned at multiple locations about the orthosis to restrict movement of the joint in a number of directions. For example, as shown in FIG. 1A-B, the orthotic device 100 includes a motion resisting mechanism 108 that includes a plurality of springs positioned on the anterior side of the lower leg. The springs apply a tension/force in a dorsiflexion direction, i.e., forcing the top of the foot up in a direction towards the user's knee. Alternatively, as shown in FIG. 1C, the motion resisting mechanism 108 is positioned on the posterior side of a user's lower leg when the user wears the orthotic device 100. In this configuration, the motion resisting mechanism 108 can apply a force/tension in a plantarflexion direction, i.e., forcing the top of the foot in a direction away from the user's knee. This is particularly useful for users who may need a different flex in one direction than the other (e.g., calf is stronger than shin).

In some embodiments, the orthotic device can include one or more sensors for measuring the force applied to the joint by the motion resisting mechanism. For example, a force/torque sensor 102a can be used to measure the torque applied by the motion resisting mechanism. By allowing a clinician to determine (1) the force applied by the motion resisting mechanism, and (2) how the user responds to that force, a clinician can fit the user with an orthosis having a stiffness better suited for that patient.

In some embodiments, the orthotic device 100 further comprises a reaction arm 106 coupled to the orthosis 110 and in mechanical communication with the motion resisting mechanism 108. The reaction arm 106 may include at least one sensor 102a, 102f The reaction arm may be coupled to the lateral face of the sensor (e.g., resistive force sensor 102a) and project to the sagittal plane of the user's leg—either anterior or posterior depending on the resistance mode—and allows the sensor(s) to collect data indicative of a user's response to resistive force applied by the springs. The motion resisting mechanism 108 may be coupled to the reaction arm 106. The reaction arm 106 may be adjustable between a first position in which the motion resisting mechanism 108 is configured to restrict movement of the joint in at least a first direction and a second position in which the motion resisting mechanism is configured to induce a second tension in which the motion resisting mechanism is configured to restrict movement of the joint in at least a second direction.

In some embodiments, the orthosis 110 can be modular and adjustable to fit a plurality of different-sized users. For example, one or more adjustable stirrups 112 and/or one or more adjustable one closure/suspension features 114 may provide modularity for the orthosis 110. The stirrup(s) 112 may have a first end and a second end, the first end coupled to the orthosis 110 at a position proximate the joint of the user, the second end coupled to the orthosis 110 at a position proximate the first side of the joint of the user. The stirrup(s) 112 may be the upper attachment point and the one closure/suspension feature(s) 114 may serve as the lower attachment point. The one closure/suspension feature(s) 114 may be configured to secure the first side of the joint of the user, e.g., calf/shin of user, to the orthosis 110. Both the one closure/suspension feature(s) 114 and stirrup(s) 112 can be height-adjustable, allowing: 1) modification of the fit of the orthosis 110 based on each user's anatomy; 2) normalization of the moment arm of force application to the user; 3) accommodation of various motion resisting mechanisms 108, e.g., extension springs of different free (i.e., unstretched) lengths; and 4) tune the angle-torque to a desired setting.

The orthotic devices 100 of the present invention can be made from many different materials. The uprights of the orthotic device 100 may be composed of bidirectional carbon fiber weave. The uprights serve as key structural components as well as a means of cable management. The uprights may have a tubular design that allows the wires and connectors to be securely and elegantly obscured within the frame of the device. The one closure/suspension feature(s) 114 and stirrup(s) 112 may be made of a carbon composite. The carbon composite provides high modulus and low weight. As a result, the orthotic device 100 can weigh between 0.6 kg and 0.9 kg without sensors and when fully equipped, respectively. The low weight maximizes transfer of load from motion resisting mechanism to user while minimizing obtrusiveness and gait perturbation. The angled joints which can be inserted into either side of the stirrup 112 may be milled out of aluminum 6061-T6, while the custom elbow joints may be made of acrylonitrile butadiene styrene (ABS) plastic.

In some embodiments, the orthotic device 100 can comprise a transmitter configured to output at least a portion of the data collected by the sensor(s) 102a-f to a second device remote from the orthotic device. The remote device can be CPU, smartphone, tablet, and the like. The remote device can be located close to the orthotic device 100 or far away. Transmittal can occur wirelessly or through wired connection. Transmittal may involve uploading and/or downloading data to a removable storage device. Transmittal may also involve collecting and storing data for later retrieval by another device. The at least a portion of data can be sent via the internet, intranet, Bluetooth, or may be stored on a cloud. The transmitter may receive a signal from the second device to begin output of at least a portion of data gathered by the sensor(s). Upon receipt of the signal, the transmitter may begin outputting for transmission at least a portion of the data gathered by the sensor(s). Transmittal of at least a portion of the data may occur in real-time. The transmitter may receive a signal from the second device indicating an end of transmission. Upon receipt of the signal to end transmission, the transmitter may end the output of at least a portion of the data to the second device.

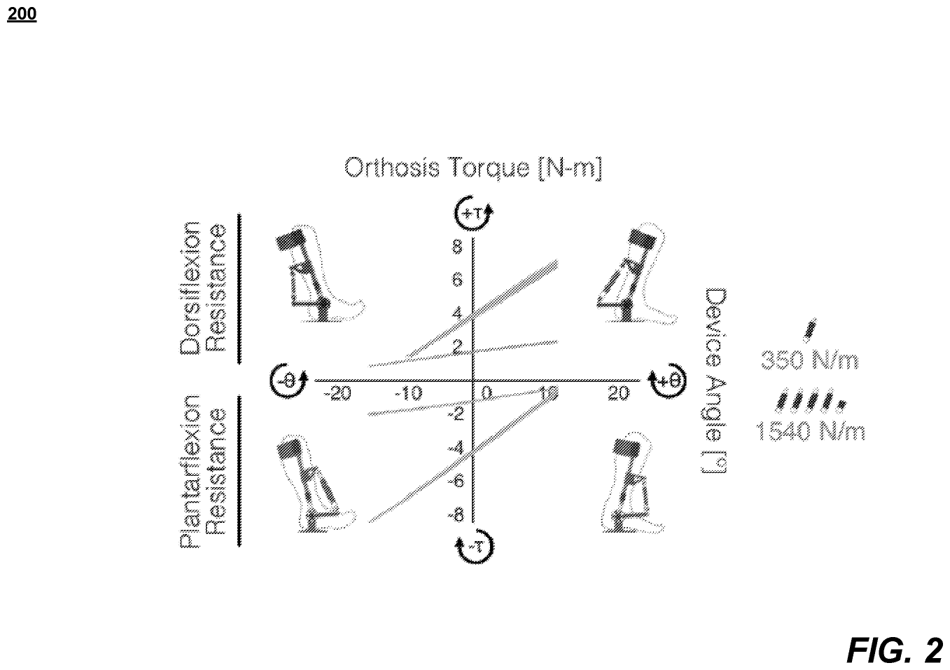

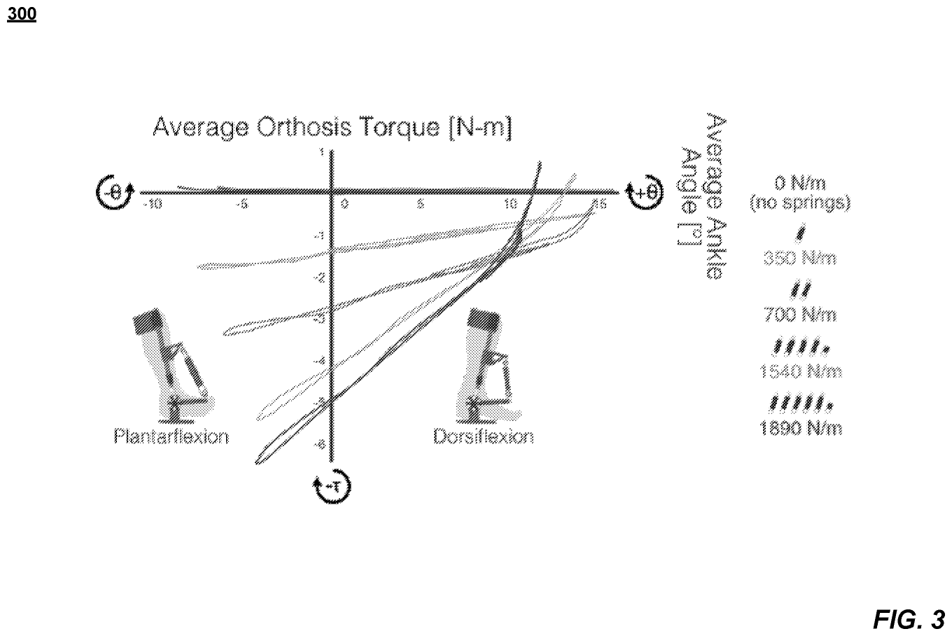

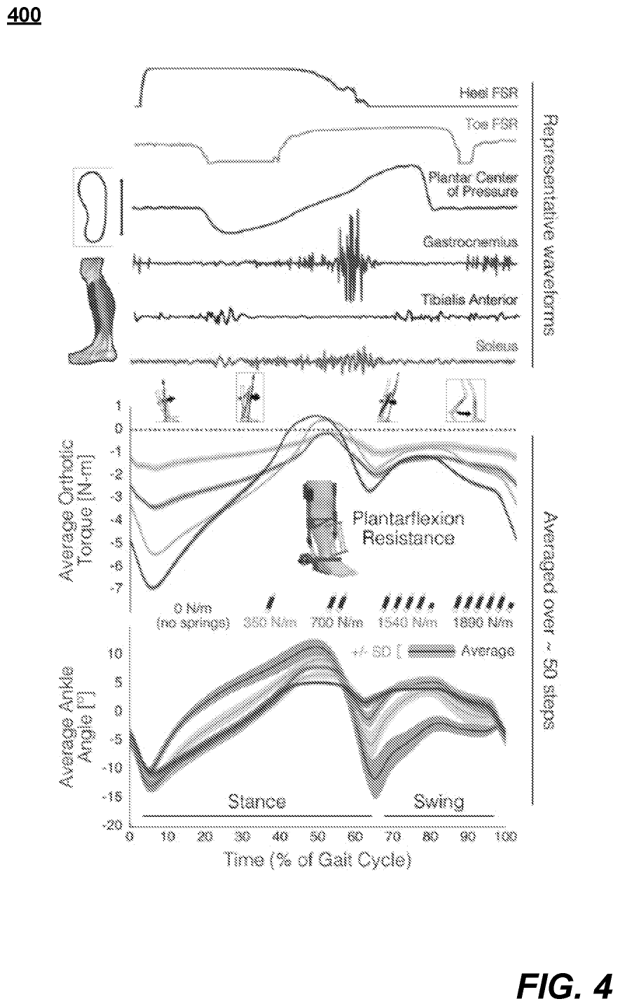

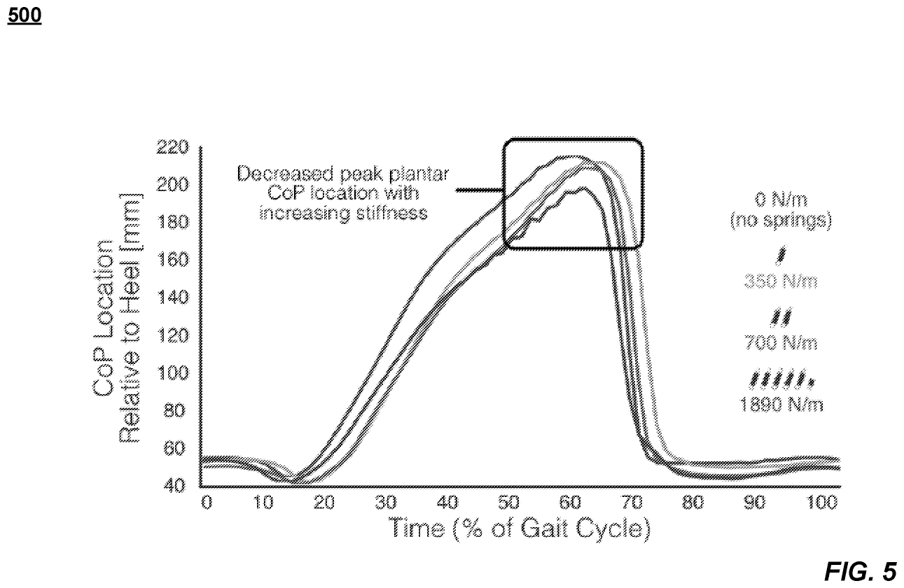

In some embodiments, once data is received by the remote device, the remote device can plot the various data collected by the orthotic device. Some of the plots include: an orthotic torque versus angle relationship in plantarflexion and dorsiflexion resistance at two stiffness conditions (350 N/m and 1540 N/m), as shown in FIG. 2; an orthotic torque versus angle relationship in plantarflexion resistance at five stiffness conditions (0 N/m, 350 N/m, 700 N/m, 1540 N/m, and 1890 N/m), as shown in FIG. 3; results of plantarflexion resistance for a single step versus approximately fifty steps, as shown at FIG. 4; and average plantar center-of-pressure location in response to increased orthotic resistance, as shown at FIG. 5. One of more of these plots can be useful for a clinician to diagnose a user and correctly fit that user for an orthosis.

Turning to the drawings, FIG. 1 is a front perspective view of an orthotic device 100 according to an embodiment, which includes a reaction arm 106 having a force/torque sensor 102a and an optical encoder 104. The orthotic device 100 may also include a passive orthosis 110, wherein at least a first portion of the orthosis is configured to be positioned on a first side of a joint of a user and at least a second portion of the orthosis is configured to be positioned on a second side of the joint of a user. The orthotic device 100 may include at least one sensor 102a-f (e.g., the force/torque sensor 102a) coupled to the orthosis 110 and configured to collect data indicative of a user's response to resistive force applied to the joint. The at least one sensor 102a-f may be configured to collect data indicative of a user's response to resistive force applied to the joint by collecting at least one of a user's response to resistive force applied to the first portion of the orthosis and a user's response to resistive force applied to the second portion of the orthosis. Also, the at least one sensor 102a-f may collect data indicative of foot-ground placement of the user during a movement by the user, range of motion of the joint during a movement by the user, and/or pressures applied to the orthosis by portions of the user during a movement by the user. The at least one sensor 102a-f may also be configured to configured to collect data indicative of a user's response to a resistive force applied to the joint at multiple times during a movement by the user.

The orthotic device 100 may further comprise at least one motion resisting mechanism 108 (e.g., a spring mechanism) configured to restrict movement of the joint in at least one direction. The motion resisting mechanism 108 may include any motion resisting mechanism known in the art, including, but not limited to, springs, hydraulic systems, flexible materials (e.g., rubber bands), magnetic systems, and the like. The motion resisting mechanism 108 may be adjustable to vary the magnitude of restriction in at least the first direction that the joint of the user moves. In some embodiments, the motion resisting mechanism 108 comprises a plurality of springs mounted in parallel with each other to provide a resistance to ankle rotation in at least one of plantarflexion and dorsiflexion. The motion resisting mechanism 108 may be coupled to the orthosis 110 about many positions of the orthosis, including, but not limited to, an anterior, posterior, medial, or lateral side of the user, or positions therebetween.

In some embodiments, the reaction arm 106 may be coupled to the orthosis 110 such that the reaction arm 106 is in mechanical communication with the motion resisting mechanism 108 (e.g., a spring mechanism). The reaction arm 106 may include at least one sensor 102a-f (e.g., force/torque sensor 102a). The reaction arm 106 may be adjustable between a first position in which the motion resisting mechanism is configured to induce a first tension in at least the first direction that the joint of the user moves and a second position in which the motion resisting mechanism (e.g., a spring mechanism) is configured to induce a second tension in at least a second direction that the joint of the user moves. In some embodiments, the orthosis 110 is modular and adjustable to fit a plurality of users. The orthosis may be modular and adjustable to allow the at least one sensor 102a-f (e.g., force/torque sensor 102s) to collect data indicative of the torque applied to the joint by the user in a plurality of directions.

According to some embodiments, the orthotic device 100, further comprises a transmitter (not shown) coupled to the orthosis 110. The transmitter can be configured to output at least a portion of the data collected by the at least one sensor 102a-f to a second device remote from the orthotic device. The orthosis 110 may also include at least one stirrup 112 having a first end and a second end. The first end can be coupled to the orthosis at a position proximate the joint of the user, and the second end can be coupled to the orthosis at a position proximate the first side of the joint of the user. The orthosis 110 may further comprise at least one closure/suspension feature 114 configured to secure the first side of the joint of the user to the orthosis. In some embodiments, the at least one sensor 102c is coupled to the at least one closure/suspension feature 114. The orthotic device 100 may be wearable by the user.

FIG. 2 is a graph illustrating benchtop testing results: orthotic torque versus angle relationship in both resistance modes for two stiffness conditions according to an embodiment of the invention. The two stiffness conditions are 350 N/m and 1540 N/m. The two curves above the x-axis were generated with the orthotic device 100 in its dorsiflexion resistance mode (i.e., extension springs mounted to the posterior of the calf), while the curves below the y-axis correspond to the device in its plantarflexion resistance mode. In the angle regime of interest (−15° to 15°), the device applies torque in a linear fashion and does nearly equivalently in both modes. The slopes of the curves corresponding to each stiffness condition appear similar in both resistance modes, suggesting that the orthotic device's 100 capacity for directional stiffness allows for highly comparable investigations of the effect of orthotic resistance in both directions.

FIG. 3 is a graph illustrating orthotic torque versus ankle angle relationship in plantarflexion resistance gait study in five stiffness conditions according to an embodiment of the invention. Applying torque to the user linearly, the curves represent the ensemble average of individual torque versus angle curves across approximately fifty steps. As shown in FIG. 3, the motion resisting mechanism may be configured to provide a force to the joint of the user in a first direction that the joint of the user moves, the force being absent (x axis) or at least one of 350 N/m, 700 N/m, 1540 N/m, and 1890 N/m (from top to bottom, respectively).

FIG. 4 is a graph illustrating plantarflexion resistance at a plurality of steps according to an embodiment of the invention. The top six waveforms are representative of sensor outputs for a single step. The bottom two plots are comprised of orthotic torque and ankle angle waveforms averaged across approximately 50 steps, respectively, plotted against the percentage of one gait cycle.

FIG. 5 is a graph illustrating average plantar center-of-pressure location as orthotic resistance increases according to an embodiment of the invention. The center-of-pressure (CoP) location is defined as the lengthwise distance along the right foot beginning at the back of the heel, measured in millimeters. Peak CoP location decreased progressively from 216 to 213.5 to 210.1 and finally to 202.3 mm for each increasing stiffness challenge, suggesting a diminished ability to transfer plantar forces towards the forefoot, likely resulting in a decrease in the anatomical torque produced by the user at the ankle joint.

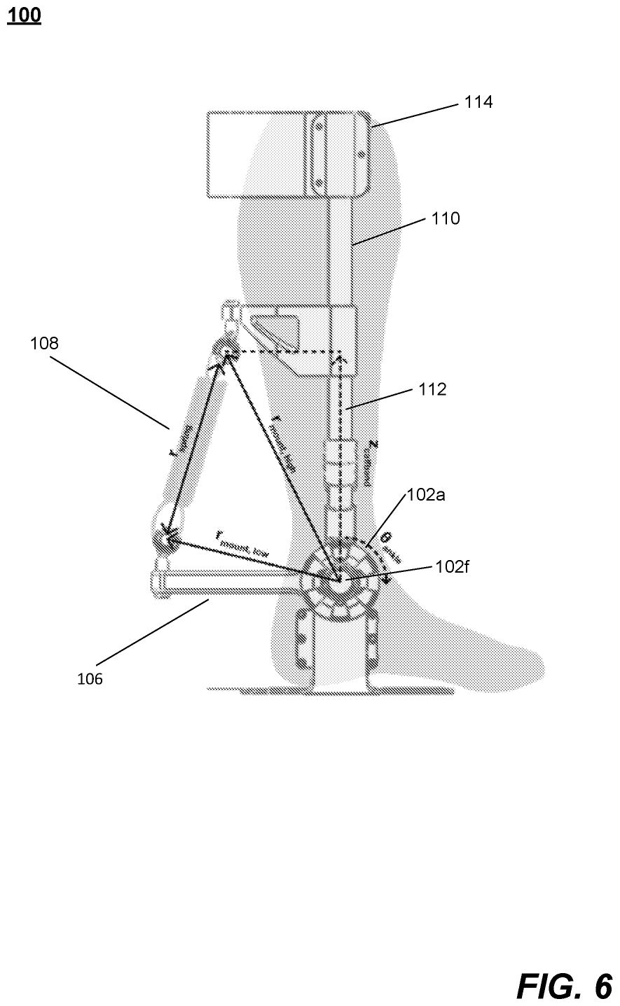

FIG. 6 illustrates the geometric parameters used in Equations (1) through (3) below to construct the mathematical model of the torque versus angle relation according to an embodiment of the invention.

$$\begin{matrix}{\vec{r}}_{mount , high} = f ( {θ}_{ankle} , {z}_{calfband} ) & ( 1 ) \\ {\vec{r}}_{spring} = {\vec{r}}_{mount , low} - {\vec{r}}_{mount , high} & ( 2 ) \\ τ = {\vec{r}}_{mount , low} \times \{ {k}_{spring} [ {\vec{r}}_{spring} - \frac{{\vec{r}}_{spring}}{‖ {\vec{r}}_{spring} ‖} {L}_{0} ] \} & ( 3 )\end{matrix}$$

Equation (1) suggests that r_{mount,high}, the vector from the center of rotation at the ankle joint to the upper attachment point of the spring(s), is simply a (trigonometric) function of Z_{calfband}, which is the adjustable calf band height setting, and θₐₙₖₗₑ, which is the rotation of the user's ankle joint, measured clockwise from the positive vertical axis. Equation (2) defines r_{spring} as the vector difference between r_{mount,high} and r_{mount,low}, which is the vector from the center of the ankle joint to the lower attachment point of the spring mechanism. The final calculation relating torque and ankle angle is shown in (3), where τ is the calculated orthotic torque applied to the user, k_{spring} is the stiffness condition imposed, and L0 is the free length of the spring(s) used. The term within the curly braces represents the force developed in the extension spring(s) as a result of lengthening, calculated using Hooke's Law, though k_{spring} is taken not as a constant but rather as a function of spring deflection (the term within the square brackets), determined experimentally.

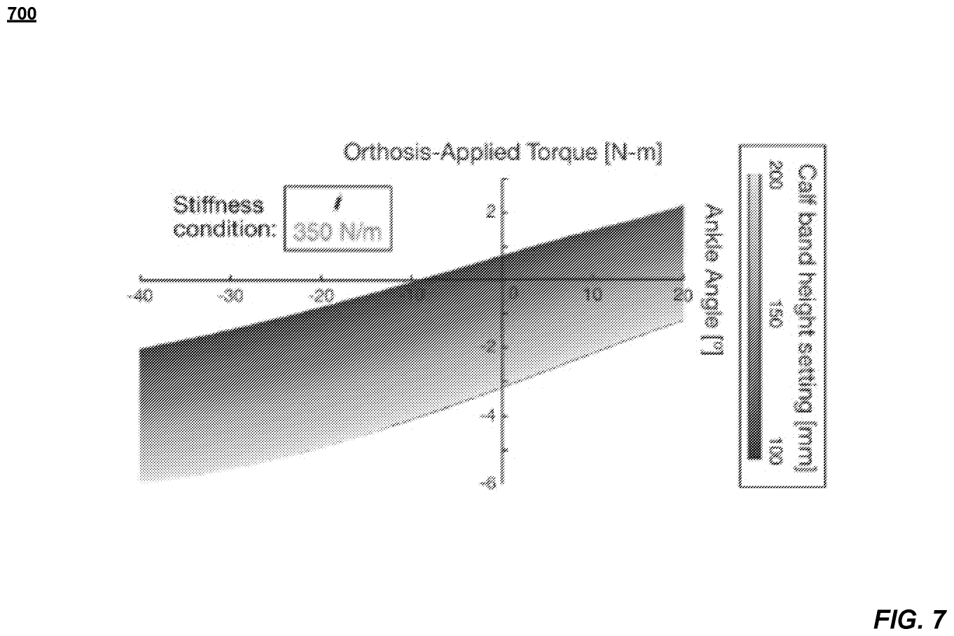

FIG. 7 is a graph illustrating model torque versus angle output for a range of calf band height settings according to an embodiment of the invention. Applying a stiffness of 350 N/m in a plantarflexion resistance direction, the individual isochromatic curve corresponds to a specific height setting. A height setting of 160 mm provided a good balance of minimizing zero-angle torque and mitigating nonlinearity at high angles of dorsiflexion (not shown).

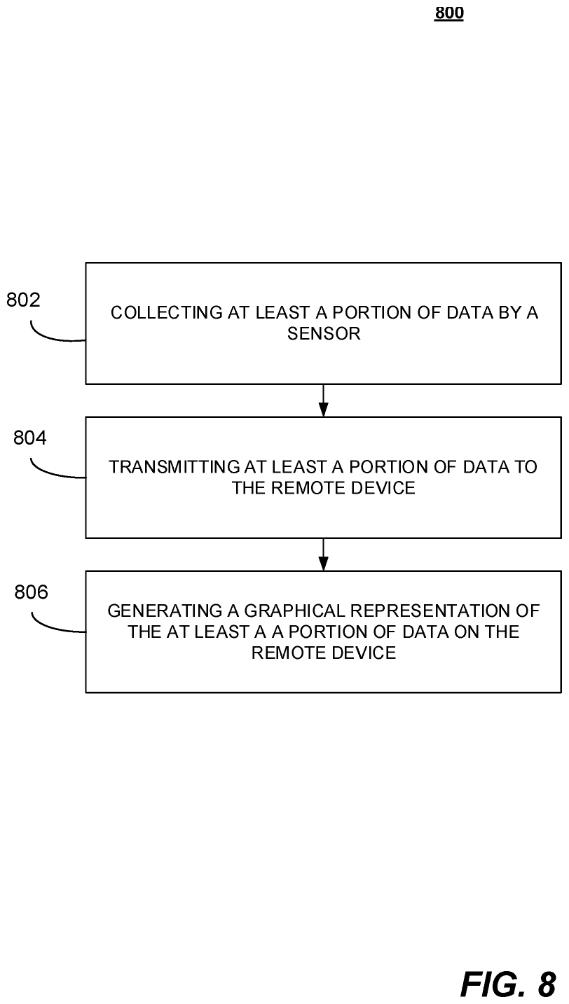

FIG. 8 illustrates an example flow chart of a for transmitting data collected by at least one sensor of a passive orthosis, the passive orthosis positioned about a joint of a user according to an embodiment of the invention. The method may include the use of an orthotic device comprising at least one sensor, a transmitter, and a remote device. At 802, the at least one sensor (e.g., force/torque sensor 102) of the passive orthosis 100 collects data indicative of a user's response to a resistive force applied to the joint. In some embodiments, the sensor receives a signal from the remote device comprising an indication to start an output of the at least a portion of data and an indication to stop the output of the at least a portion of data. At 804, the orthotic device 100 transmits at least a portion of the data collected by the at least one sensor of the passive orthosis to a remote device. In some embodiments, the transmission of the at least a portion of the data collected by the at least one sensor to the remote device is performed in real-time as the user moves the joint. At 806, a graphical representation of the at least a portion of the data collected by the at least one sensor is generated on the remote device.

Although the invention has been described by reference to specific embodiments, it should be understood that numerous changes may be made within the spirit and scope of the inventive concepts described. Accordingly, it is intended that the invention not be limited to the described embodiments, but that it have the full scope defined by the language of the following claims. Further, the features illustrated or described in connection with one example embodiment may be combined with the features of other embodiments. Such modifications and variations are intended to be included within the scope of the present disclosure. Further, in the present disclosure, like-numbered components of the embodiments generally have similar features and/or purposes.

1. An orthotic device comprising:
a passive orthosis having a first portion and a second portion;
a sensor;
a motion resisting mechanism; and
a reaction arm;
wherein the first portion of the passive orthosis is configured to be positionable on a first side of a joint of a user;
wherein the second portion of the passive orthosis is configured to be positionable on a second side of the joint;
wherein the sensor is configured to collect data indicative of the user's response to a resistive force applied to the joint;
wherein the motion resisting mechanism is configured to restrict movement of the joint in a direction; and
wherein the reaction arm is in mechanical communication with the motion resisting mechanism.

2. The orthotic device of claim 1, wherein the data indicative of the user's response to the resistive force applied to the joint includes data selected from the group consisting of data indicative of:
foot-ground placement of the user during a movement by the user;
a motion of the joint during a movement by the user;
a resistive force applied to the joint during a movement by the user; and
a pressure applied to the passive orthosis during a movement by the user.

3.-6. (canceled)

7. The orthotic device of claim 1, wherein the motion resisting mechanism comprises a spring.

8. The orthotic device of claim 1 further comprising another sensor configured to collect data indicative of a force applied by the motion resisting mechanism.

9. (canceled)

10. The orthotic device of claim 1, wherein the motion resisting mechanism is adjustable to vary a magnitude of a force applied by the motion resisting mechanism.

11. The orthotic device of claim 1, wherein the motion resisting mechanism is coupled to the passive orthosis on an anterior side of the first side of the joint.

12. The orthotic device of claim 1, wherein the motion resisting mechanism is coupled to the passive orthosis on a posterior side of the first side of the joint.

13. (canceled)

14. The orthotic device of claim 1, wherein the reaction arm is adjustable between a first position in which the motion resisting mechanism is configured to resist movement of the joint in a first direction and a second position in which the motion resisting mechanism is configured to restrict movement of the joint in a second direction different than the first direction.

15. The orthotic device of claim 1, wherein the passive orthosis is modular and adjustable.

16. The orthotic device of claim 1 further comprising a transmitter configured to output at least a portion of the data collected by the sensor to a second device remote from the orthotic device.

17. The orthotic device of claim 1, wherein the passive orthosis further comprises a stirrup having a first end and a second end;
wherein the first end of the stirrup is coupled to the passive orthosis at a position proximate the joint; and
wherein the second end of the stirrup is coupled to the passive orthosis at a position proximate the first side of the joint.

18. The orthotic device of claim 1, wherein the passive orthosis further comprises a closure/suspension configured to secure the first side of the joint to the passive orthosis.

19.-20. (canceled)

21. A method comprising:
wearing a passive orthosis having a first portion positioned on a first side of a joint and a second portion positioned on a second side of the joint;
applying a resistive force to the joint with a motion resisting mechanism that is in mechanical communication with a reaction arm; and
collecting data indicative of the user's response to the resistive force applied to the joint.

22. The method of claim 21 further comprising:
storing at least a portion of the collected data on a memory;
transmitting at least a portion of the collected data in real-time as the joint moves; and
generating a graphical representation of at least a portion of the transmitted data.

23.-24. (canceled)

25. An orthotic device comprising:
a passive orthosis positionable proximate at least one joint of a user;
a first sensor;
a second sensor;
a transmitter;
a motion resisting mechanism:
configured to restrict movement of the joint in at least one direction via a resisting force; and
adjustable to vary a magnitude of the resisting force applied by the motion resisting mechanism; and
a reaction arm in mechanical communication with the motion resisting mechanism;
wherein the first sensor is configured to collect data indicative of the user's response to the resistive force applied to the joint by the motion resisting mechanism;
wherein the second sensor is configured to collect data indicative of the resisting force applied by the motion resisting mechanism; and
wherein the transmitter is configured to output at least a portion of the data collected by one or both of the first sensor and the second sensor to a remote device remote from the orthotic device.

26. The orthotic device of claim 25, wherein:
the passive orthosis comprises:
a first portion configured to be secured to a lower leg of a user between an ankle and a knee of the user; and
a second portion configured to be secured to a foot of the user;
the first sensor is coupled to the passive orthosis and configured to collect the data indicative of the user's response to the resistive force applied to the ankle; and
the motion resisting mechanism is configured to restrict movement of the ankle in at least one of a plantarflexion direction and a dorsiflexion direction by applying the resistive force to the ankle.

27.-30. (canceled)

31. The orthotic device of claim 26, wherein the motion resisting mechanism comprises at least one spring.

32.-34. (canceled)

35. The orthotic device of claim 26, wherein the motion resisting mechanism is coupled to the passive orthosis on an anterior side of a first side of the ankle.

36. The orthotic device of claim 26, wherein the motion resisting mechanism is coupled to the passive orthosis on a posterior side of a first side of the ankle.

37. (canceled)

38. The orthotic device of claim 26, wherein the reaction arm is adjustable between a first position on the anterior side of the ankle in which the motion resisting mechanism is configured to restrict movement of the ankle in the plantarflexion direction and a second position on the posterior side of the ankle in which the motion resisting mechanism is configured to restrict movement of the ankle in the dorsiflexion direction.
